(19) United States
(12) Patent Application Publication
(10) Pub. No.: US 2006/0240005 A1
(43) Pub. Date: Oct. 26, 2006

(54) Methods and compositions for increasing the efficiency of therapeutic antibodies using alloreactive natural killer cells

(75) Inventor: Andréa Velardi, Perugia (IT)

Correspondence Address: SALIWANCHIK LLOYD & SALIWANCHIK;A PROFESSIONAL ASSOCIATION, PO BOX 142950, GAINESVILLE, FL, 32614-2950, US

(73) Assignee: Universita Degli Studi di Perugia, Perugia (IT)

(21) Appl. No.: 10/565,761

(22) PCT Filed: Jul. 23, 2004
(86) PCT No.: PCT/IB04/02637

§ 371 Date: Jan. 24, 2006

Related U.S. Application Data
(60) Provisional application No. 60/489,509, filed on Jul. 24, 2003.

(51) Int. Cl. A61K 39/395; A61K 35/14

(52) U.S. Cl. 424/144.1; 424/93.7

(57) ABSTRACT

The present invention relates, generally, to methods and compositions for increasing the efficiency of therapeutic antibodies. Their efficiency is enhanced through the increase of the ADCC mechanism. More particularly, the invention relates to the use of a therapeutic antibody in combination with alloreactive natural killer cells in order to enhance the efficiency of the treatment with therapeutic antibody in human subjects.

### FIELD OF THE INVENTION

The present invention relates, generally, to methods and compositions for increasing the efficiency of therapeutic antibodies. More particularly, the invention relates to the use of a therapeutic antibody in combination with alloreactive natural killer cells in order to enhance the efficiency of the treatment in human subjects, in particularly through an increase in ADCC mechanism.

### BACKGROUND OF THE INVENTION

Various therapeutic strategies in human beings are based on the use of therapeutic antibodies. This includes, for instance, the use of therapeutic antibodies developed to deplete target cells, particularly diseased cells such as vially-infected cells, tumor cells or other pathogenic cells. Such antibodies are typically monoclonal antibodies, of IgG species, typically with human IgG1 or IgG3 Fc portion. These antibodies can be native or recombinant antibodies, humanized mice antibodies (i.e. comprising functional domains from various species, typically Fc portion of human or non human primate origin, and variable region or complementary determining region (CDR) of mice origin). Alternatively, the monoclonal antibody can be fully human through immunization in human Ig locus transgenic mice or obtained through cDNA libraries derived from human cells. A particular example of such therapeutic antibodies is rituximab (Mabthera®, Rituxan®), which is a chimeric anti-CD20 monoclonal antibody made with human γ1 and κ constant regions (therefore with human IgG1 Fc portion) linked to murine variable domains conferring CD20 specificity. In the past few years, rituximab has considerably modified the therapeutical strategy against B lymphoproliferative malignancies, particularly non-Hodgkin's lymphomas (NHL). Other examples of humanized IgG1 antibodies include alemtuzumab (Campath-1H®), which is used in the treatment of B cell malignancies or trastuzumab (Herceptin®), which is used in the treatment of breast cancer. Additional examples of therapeutic antibodies under development are disclosed in the art.

The mechanism of action of therapeutic antibodies is still a matter of debate. Injection of antibodies leads to depletion of cells bearing the antigen specifically recognized by the antibody. This depletion can be mediated through at least three mechanism: antibody mediated cellular cytotoxicity (ADCC), complement dependant lysis, and direct antitumor inhibition of tumor growth through signal given via the antigen targeted by the antibody.

While these antibodies represent a novel efficient approach to human therapy, particularly for treatment of tumors, they do not always exhibit a strong efficacy. For instance, while rituximab, alone or in combination with chemotherapy was shown to be effective in the treatment of both low-intermediate and high-grade NHL, 30% to 50% of patients with low grade NHL have no clinical response to rituximab. It has been suggested that the level of CD20 expression on lymphoma cells, the presence of high tumor burden at the time of treatment or low serum rituximab concentrations may explain the lack of efficacy of rituximab in some patients. Nevertheless, the actual causes of treatment failure remain largely unknown. There is therefore a need in the art for increasing the efficiency of the therapeutic antibodies.

### SUMMARY OF THE INVENTION

The present invention discloses novel approaches to enhance the efficacy of the therapeutic antibodies. These approaches are based on the increase of the ADCC mechanism in vivo, when therapeutic antibodies are injected. Preferably, the increase of the ADCC mechanism is achieved by the administration of alloreactive natural killer (NK) cells.

More specifically, the invention discloses methods of treatment of a subject in which alloreactive human NK cells are co-administered with the therapeutic antibody or a fragment thereof to the subject. As demonstrated by the inventors in human subjects, alloreactive NK cells display much more potent ADCC activity compared to autologous NK cells. By alloreactive, it is meant that NK cells do not display a KIR inhibitory receptor compatible with the HLA of a host. The inventors demonstrate here that the efficiency of a therapeutic antibody can be greatly enhanced by the co-injection of selected, alloreactive NK.

The invention concerns a pharmaceutical composition comprising a therapeutic antibody or a fragment thereof and alloreactive human natural killer cells. The invention also concerns a kit comprising a therapeutic antibody or a fragment thereof and alloreactive human natural killer cells.

The invention concerns the use of alloreactive NK cells for increasing the efficiency of a treatment with a therapeutic antibody, or for increasing ADCC in a subject submitted to a treatment with a therapeutic antibody or a fragment thereof.

The invention also concerns the use of alloreactive natural killer cells and of a therapeutic antibody or a fragment thereof for the preparation of a drug for treating a disease. More particularly, the treatment of the disease requires the depletion of the targeted cells, preferably the diseased cells such as virally-infected cells, tumor cells or other pathogenic cells, including allogenic immunocompetent cells. Preferably, the disease is a cancer, infectious or immune disease. More preferably, the disease is selected from the group consisting of a cancer, an auto-immune disease, an inflammatory disease, a viral disease. The disease also concerns a graft rejection, more particularly allograft rejection, and graft versus host disease (GVHD).

The present invention also comprises a method for reducing the dosage of a therapeutic antibody, e.g. an antibody that is bound by CD16. For example, co-administration of a therapeutic antibody and alloreactive natural killer cells allows a lower dose of the therapeutic antibody to be used. Therapeutic antibodies can be used in such a context at a 20%, 30%, 40%, 50%, 60%, 70%, 80%, 90%, or lower dose than the recommended dose in the absence of the compound.

In addition, the invention provides a method for determining a therapeutically-effective, reduced dose of a therapeutic antibody, e.g., an antibody bound by CD16, the method comprising i) co-incubating a first concentration of the therapeutic antibody with target cells and NK cells, and in the absence of alloreactive natural killer cells; ii) co-incubating a second, lower concentration of the therapeutic antibody with target cells, with NK cells, and in the presence of alloreactive natural killer cells; iii) determining if the depletion of target cells observed in step ii) is as great as the depletion observed in step i). If it is observed that step ii) is as efficacious as step i), then the relative concentrations of the alloreactive natural killer cells and the therapeutic antibody can be varied, and depletion observed, in order to identify different conditions that would be suitable for use in a given patient, e.g., maximizing target cell depletion, lowered dose of therapeutic antibody, or lowered dose of the alloreactive natural killer cells, depending on the particular needs of the patient.

In a particular aspect, the invention concerns a method of treatment of a subject in need thereof comprising:
a) administering to said subject alloreactive natural killer cells; and,
b) administering to said subject a therapeutic antibody.

Said therapeutic antibody is capable of forming an immune complex. Preferably, said therapeutic antibody can be bound by CD16 receptor present on NK cells, preferably through its Fc region. In a preferred embodiment, the therapeutic antibody has a human or non human primate IgG1 or IgG3 Fc portion. Preferably, the therapeutic antibody is a monoclonal antibody or a fragment or a derivative thereof, more preferably a humanized, human or chimeric antibody. In a particular embodiment, the therapeutic antibody is rituximab. Said fragment or a derivative thereof is preferably selected from a Fab fragment, a Fab′2 fragment, a CDR and a ScFv.

### LEGEND TO THE FIGURES

FIG. 1: ADCC by alloreactive or non alloreactive human NK cells on EBV transformed B cells. 5 ADCC is much more potent with alloreactive NK cells.

FIG. 2: Rescue of NOD-SCID mice from EBV lymphoproliferation disease by alloreactive NK cells and Rituxan. Rituxan alone has no effect in NOD-SCID. Alloreactive NK rescue NOD SCID from leukaemia induced by and EBV transformed cancer cell line when a ratio of 1 NK cell versus 25 EBV target cell is used. When a ratio of 1 NK versus 100 EBV target is used, the mice are not rescued. When combined with Rituxan, the latter ratio of effectors to target is sufficient to rescue the mice.

### DETAILED DESCRIPTION OF THE INVENTION

The present invention provides a mean to increase the efficiency of the therapeutic antibodies. The invention more specifically discloses that the use of alloreactive NK cells having certain phenotype or properties can significantly increase the efficiency of therapeutic antibodies. Indeed, the inventors demonstrate that the efficiency of a therapeutic antibody can be greatly enhanced by the co-injection of alloreactive natural killer cells.

Therefore, the invention concerns a method of treatment of a disease in a subject in need thereof comprising:
a) administering to said subject alloreactive natural killer cells; and,
b) administering to said subject a therapeutic antibody.

Said therapeutic antibody can be bound by CD16, preferably through its Fc region. Optionally, the method comprises an additional step of transplanting an allogeneic graft into said subject. Preferably, said allogeneic graft is a hematopoietic graft. Optionally, said hematopoietic graft is a bone marrow transplant.

More particularly, the treatment of the disease requires the depletion of the targeted cells, preferably the diseased cells such as virally-infected cells, tumor cells or other pathogenic cells, including allogenic immunocompetent cells. Preferably, the disease is a cancer, infectious or immune disease. More preferably, the disease is selected from the group consisting of a cancer, an auto-immune disease, an inflammatory disease, a viral disease. The disease also concerns a graft rejection, more particularly allograft rejection, and graft versus host disease (GVHD).

Said diseases include neoplastic proliferation of hematopoietic cells. Optionally, said diseases are selected from the group consisting of lymphoblastic leukemia, acute or chronic myelogenous leukemia, Hodgkin's lymphoma, Non-Hodgkin's lymphoma, myelodysplastic syndrome, multiple myeloma, and chronic lymphocytic leukemia. Said diseases also include ENT cancers, colorectal cancers, breast cancer, epithelial cancer. Said diseases include CMV infection, and hepatitis B. Said diseases include Crohn disease, rheumatoid arthritis, asthma, psoriasis, multiple sclerosis, or diabetes.

Preferably, said therapeutic antibody has a human or non human primate IgG1 or an IgG3 Fc portion, particularly a monoclonal antibody or a fragment thereof, further preferably a human, humanized or chimeric antibody or a fragment thereof for instance rituximab.

It is intended that alloreactive natural killer cells can be administered to the subject before, simultaneously with or, after the administration of the therapeutic antibody. Preferably, the therapeutic antibody is administrated within the 5 days period around the administration of the natural killer cells, more preferably within the 2 days period. Preferably, the therapeutic antibody is administrated before or simultaneously with the alloreactive natural killer cells.

Preferably, said alloreactive natural killer cells comprise at least 5% of alloreactive NK cells against the recipient cells among its NK cells content, preferably at least 20 or 30%, more preferably at least 40 or 50%, still more preferably at least 60, 70 or 90%. Optionally, essentially all the NK cells are allorective against the recipient cells.

Preferably, said subject is treated under myelo-reductive regimen or an immunosuppressive treatment before the administration of the alloreactive natural killer cells, optionally myelo-ablative regimen.

In a further aspect, the invention concerns a method of increasing ADCC in a subject receiving therapeutic antibody treatment, said method comprising administering to said subject prior to, simultaneously or after the administration of said therapeutic antibody an amount of alloreactive natural killer cells sufficient to increase ADCC. Said therapeutic antibody can be bound by CD16 on NK cells, preferably through its Fc region. Preferably, said therapeutic antibody has a human or non human primate IgG1 or an IgG3 Fc portion, particularly a monoclonal antibody or a fragment thereof further preferably a chimeric, human or humanized antibody or a fragment thereof, for instance rituximab.

In an additional aspect, the invention concerns a method of increasing the efficiency of a therapeutic antibody, said method comprising administering to said subject prior to, simultaneously or after the adminstration of said therapeutic antibody an amount of alloreactive natural killer cells sufficient to increase the efficiency of said therapeutic antibody. Said therapeutic antibody can be bound by CD16, preferably through its Fc region. Preferably, said therapeutic antibody has a human or non human primate IgG1 or an IgG3 Fc portion, particularly a monoclonal antibody or a fragment thereof, further preferably a human, humanized or chimeric antibody or a fragment thereof, for instance rituximab.

The invention concerns a method of treating a subject having hematologic disorder comprising transplanting a allogeneic graft into said subject so as to treat said disorder, the improvement comprising administering to said subject an effective amount of alloreactive donor-vs.-recipient natural killer cells and of therapeutic antibodies.

The invention contemplates a method of avoiding the tumor relapse in a subject comprising administering to the subject an effective amount of active alloreactive donor-vs-recipient human natural killer cells and of therapeutic antibodies, which are effective against the tumor relapse in combination transplanting the allogeneic graft into the subject.

Within the context of the present invention, a subject or patient includes any mammalian subject or patient, more preferably a human subject or patient.

Thereapeutic Antibody

Within the context of this invention, the term “therapeutic antibody or antibodies” designates more specifically any antibody that functions to deplete target cells in a patient. Specific examples of such target cells include tumor cells, virus-infected cells, allogenic cells, pathological immunocompetent cells (e.g., B lymphocytes, T lymphocytes, antigen-presenting cells, etc.) involved in allergies, autoimmune diseases, allogenic reactions, etc., or even healthy cells (e.g., endothelial cells in an anti-angiogenic therapeutic strategy). Most preferred target cells within the context of this invention are tumor cells and virus-infected cells. The therapeutic antibodies may, for instance, mediate a cytotoxic effect or a cell lysis, particularly by antibody-dependant cell-mediated cytotoxicity (ADCC). ADCC requires leukocyte receptors for the Fc portion of IgG (FcγR) whose function is to link the IgG-sensitized antigens to FcγR-bearing cytotoxic cells and to trigger the cell activation machinery. While this mechanism of action has not been evidenced in vivo in humans, it may account for the efficacy of such target cell-depleting therapeutic antibodies. Therefore, the therapeutic antibody is capable of forming an immune complex. For example, an immune complex can be a tumoral target covered by therapeutic antibodies. More particularly, the antibody can be bound by CD16, preferably through its Fc region. The therapeutic antibodies may by polyclonal or, preferably, monoclonal. They may be produced by hybridomas or by recombinant cells engineered to express the desired variable and constant domains. The antibodies may by single chain antibodies or other antibody derivatives retaining the antigen specificity and the lower hinge region or a variant thereof. These may be polyfunctional antibodies, recombinant antibodies, humanized antibodies, fragments or variants thereof. Said fragment or a derivative thereof is preferably selected from a Fab fragment, a Fab′2 fragment, a CDR and a ScFv. Therapeutic antibodies are specific for surface antigens, e.g., membrane antigens. Most preferred therapeutic antibodies are specific for tumor antigens (e.g., molecules specifically expressed by tumor cells), such as CD20, CD52, ErbB2 (or HER2/Neu), CD33, CD22, CD25, MUC-1, CEA, KDR, αVβ33, etc., particularly lymphoma antigens (e.g., CD20). The therapeutic antibodies have preferably human or non human primate IgG1 or IgG3 Fc portion, more preferably human IgG1.

In one embodiment, the antibodies will include modifications in their Fc portion that enhances the interaction of the antibody with NK cells during ADCC. Such modified therapeutic antibodies (“altered antibodies”) generally comprise modifications preferably, in the Fc region that modify the binding affinity of the antibody to one or more Fc?R. Methods for modifying antibodies with modified binding to one or more Fc?R are known in the art, see, e.g., PCT Publication Nos. WO 2004/016750 (International Application PCT/US2003/025399), WO 99/158572, WO 99/151642, WO 98/123289, WO 89/107142, WO 88/107089, and U.S. Pat. Nos. 5,843,597 and 5,642,821, each of which is incorporated herein by reference in their entirety.

Therapeutic antibodies identified herein, such as D2E7 (Cambridge Antibody Technology Group, plc (Cambridge, UK)/BASF (Ludwigshafen, Germany)) used to treat rheumatoid arthritis, or Infliximab (Centocor, Inc., Malvern, Pa.; used to treat Crohn's disease and rheumatoid arthritis), or the antibodies disclosed in International Patent Application PCT/US2003/025399 (which is hereby incorporated by reference in its entirety) can be modified as taught in the above and below identified applications and used for the treatment of diseases for which such antibodies are typically used. In some embodiments, the invention provides altered antibodies that have altered affinity, either higher or lower affinity, for an activating Fc?R, e.g., Fc?RIII. In certain preferred embodiments, altered antibodies having higher affinity for Fc?R are provided. Preferably such modifications also have an altered Fc-mediated effector function.

Modifications that affect Fc-mediated effector function are well known in the art (See, e.g., U.S. Pat. No. 6,194,351, which is incorporated herein by reference in its entirety). The amino acids that can be modified include but are not limited to proline 329, proline 331, and lysine 322. Proline 329 and/or 331 and lysine 322 can, preferably be replaced with alanine, however, substitution with any other amino acid is also contemplated. See International Publication No.: WO 00/142072 and U.S. Pat. No. 6,194,551 which are incorporated herein by reference in their entirety.

Thus, modification of the Fc region can comprise one or more alterations to the amino acids found in the antibody Fc region. Such alterations can result in an antibody with an altered antibody-mediated effector function, an altered binding to other Fc receptors (e.g., Fc activation receptors), an altered ADCC activity, an altered Clq binding activity, an altered complement dependent cytotoxicity activity, or any combination thereof.

In one embodiment, the antibody is specifically recognized by an Fc gamma receptor such as FCGR3A (also called CD16, FCGR3, Immunoglobulin G Fc Receptor III; IGFR3, Receptor for Fc Fragment of IgG, Low Affinity ma,; see, e.g. OMIM 146740), FCGR2A (also called CD32, CDw32, Receptor for Fc Fragment of IgG, Low Affinity IIa, FCG2, Immunoglobulin G Fc Receptor II; see, e.g. OMIM 146790); FCGR2B (also called CD32, Receptor for Fc Fragment of IgG, Low Affinity IIIb; FCGR2B, FC-Gamma-RIIB; see, e.g. OMIM 604590), FCG1RA (also called CD64; Receptor for Fc Fragment of IgG, High affinity Ia; IGFR1; see, e.g., OMIM 146760); FCGR1 fragment of IgG, High affinity Ic, Immunoglobulin G Fc receptor IC, IGFRC; see, e.g., OMIM 601503); or FCGR1B (also called CD64, Receptor for Fc Fragment of IgG, High affinity Ib; Immunoglobulin G Fc Receptor IB,; IGFRB; see, e.g., OMIM 601502).

Typical examples of therapeutic antibodies of this invention are rituximab, alemtuzumab and trastuzumab. Such antibodies may be used according to clinical protocols that have been authorized for use in human subjects. Additional specific examples of therapeutic antibodies include, for instance, epratuzumab, basiliximab, daclizmab, cetuximab, labetuzumab, seviumab, tuvurimab, palivizumab, infliximab, omalizumab, efalizuab, natalizumab, clenoliximab, etc. Other examples include anti-ferritin antibodies (US Patent Publication no. 2002/0106324), anti-p140 and anti-sc5 antibodies (WO 02/50122), and anti-KIR (NK cell killer inhibitory receptor) antibodies (The KIR receptors are described in Carrington and Norman, (The KIR Gene Cluster, May 3, 2003, available at: http://www.ncbi.nlm.nih.gov/books), the disclosures of which are incorporated herein by reference. Other examples are listed in the following table, any of which (and others) can be used in the present methods. It will be appreciated that, regardless of whether or not they are listed in the following table or described elsewhere in the present specification, any antibody that can deplete target cells, preferably by ADCC, can benefit from the present methods, and that the following table is non exhaustive, neither with respect to the antibodies listed therein, nor with respect to the targets or indications of the antibodies that are listed.

| Ab specificity | DCI | Commercial name | Typical Indications |
|---|---|---|---|
| Anti-CD20 | rituximab | MabThera ®, Rituxan ® | NHL B |
| Anti-CD20 | | Zevalin | NHL |
| Anti-CD20 | | Bexocar | NHL |
| Anti-CD52 | alemtuzumab | CAMPATH-1H ® | CLL, allograft |
| Anti-CD33 | | SMART-M195 | AML |
| Anti-CD33 | | Zamyl ™ | Acute myeloid Leukemia |
| Anti-HLA-DR | | SMART-ID10 | NHL |
| antigen | | | |
| Anti-HLA-DR | | Remitogen ™ | NHL B |
| Anti-CD22 | epratuzumab | LymphoCide ™ | NHL B |
| Anti-HER2 | | MDX-210 | Prostate and other cancers |
| Anti-erbB2 | trastuzumab | Herceptin ®, | Metastatic breast cancer |
| (HER-2/neu) | | | |
| Anti-CA125 | | OvaRex | Ovarian cancer |
| Anti-MUC1 | | TriAb | Metastatic breast cancer |
| Anti-MUC1 | | BravaRex | Metastatic cancers |
| Anti-PEM antigen | | Theragyn, Therex | Ovarian cancer, breast cancer |
| Anti-CD44 | bivatuzumab | | Head and neck cancer |
| Anti-gp72 | MAb, idiotypic | | colorectal cancer |
| | 105AD7 | | |
| Anti-EpCAM | Anti-EpCAM; | IS-IL2 | cancer |
| | MT201 | | |
| Anti-VEGF | MAb-VEGF | | metastatic NSCLC, colorectal |
| | | | cancer |
| Anti-CD18 | AMD Fab | | age-related macular degeneration |
| Anti-CD18 | Anti-CD18 | | Myocardial infarction |
| Anti-VEGF | IMC-1cl I | | colorectal cancer |
| receptor | | | |
| anti-nuC242 | nuC242-DMI | | Colorectal, gastric, and pancreatic |
| | | | cancer |
| Anti-EGFR | MAb425 | | cancer |
| Anti-EGFR | ABX-EGF | | Cancer |
| Anti-EGFR | cetuximab | | ENT and colorectal Cancers |
| (HER-1, erbB1) | | | |
| Anti-MUC-1 | | Therex ® | Breast and epithelial cancers |
| Anti-CEA | | CEAVac | Colorectal cancer |
| Anti-CEA | labetuzumab | CEA-Cide ™ | Solid tumors |
| Anti-αVβ3 | | Vitaxin | Leiomyosarcoma, colorectal and |
| | | | other cancers (anti-angiogenic) |
| Anti-KDR | | | Cancers (anti-angiogenic) |
| (VEGFR2) | | | |
| anti-VRS fusion | palivizumab | Synagis ® | Viral diseases |
| protein | | | |
| Idem | | Numax ™ | Idem |
| CMV | sevirumab | Protovir | CMV Infection |
| HBs | tuvirumab | Ostavir ™ | Hepatitis B |
| Anti-CD25 | basiliximab | Simulect ® | Prevention/treatment allograft |
| | | | rejection |
| Anti-CD25 | daclizumab | Zenapax ® | Prevention/treatment allograft |
| | | | rejection |
| anti-TNF-α | infliximab | Remicade ™ | Crohn disease, rheumatoid arthritis |
| anti-CD80 | IDEC-114 | | psoriasis |
| anti-IgE | | E-26 | Allergic asthma and rhinitis |
| anti-IgE | omalizumab | Xolair ™ | Asthma |
| anti-IgE | Rhu-mAb E25 | | Allergy/asthma |
| anti-integrin αL | efalizumab | Xanelim ™ | psoriasis |
| (CD11a, LFA-1) | | | |
| Anti-beta 2 | LDP-01 | | Stroke, allograft rejection |
| integrin | | | |
| anti-integrin αL | anti-CD11a | | psoriasis |
| (CD11a, LFA-1) | | | |
| anti-CD4 | keliximab | | GVHD, psoriasis |
| | siplizumab | | |
| | MEDI-507 | | |
| Anti-CD4 | OKT4A | | Allograft rejection |
| Anti-CD3 | OKT3 | | Allograft rejection |
| Anti-CD3 | SMART-aCD3 | | Autoimmune disease, allograft |
| | | | rejection, psoriasis |
| Anti-CD64 | | | anemia |
| anti-CD147 | | | GvHD |
| anti-integrin α4 | natalizumab | Antegren ® | Multiple Sclerosis, Crohn |
| (α4β1-α4β7) | | | |
| Anti-integrin β7 | | | Crohn, ulcerative colitis |
| Alpha 4 beta 7 | LDP-02 | | Ulcerative colitis |
| Anti-HLA-DR10 | | Oncolym | NHL |
| beta | | | |
| Anti-CD3 | | Nuvion | T cell malignancies |
| Anti-GD2 | | Trigem | Metastatic melanoma and small cell |
| ganglioside | | | lung cancer |
| Anti-SK-1 antigen | | | Colorectal and pancreatic |
| | | | carcinoma |
| anti-CD4* | clenoliximab | | |
| anti-IL-8 | ABX-IL8 | | psoriasis |
| Anti-VLA-4 | | Antegren | MS |
| Anti-CD40L | | Antova | SLE, allograft rejection |
| Anti-CD40L | IDEC-131 | | MS, SLE |
| Anti-E-selectin | CDP850 | | psoriasis |
| Anti-CD11/CD18 | Hu23F2G | | MS, stroke |
| Anti-ICAM-3 | ICM3 | | psoriasis |
| Anti-CBL | ABX-CBL | | GVHD |
| Anti-CD147 | | | |
| Anti-CD23 | IDEC-152 | | Asthma, allergies |
| Anti-CD25 | | Simulect | Allograft rejection |
| Anti-T1-ACY | ACY-110 | | Breast cancer |
| Anti-TTS | TTS-CD2 | | Pancreatic, renal cancer |
| Anti-TAG72 | AR54 | | Breast, ovarian, lung cancer |
| Anti-CA19.9 | GivaRex | | Colorectal, pancreatic, gastric |
| Anti-PSA | ProstaRex | | Prostate cancer |
| Anti-HMFG1 | R1550 | | Breast, gastric cancer |
| | pemtumomab | Theragyn | Gastric, ovarian cancer |
| Anti-hCG | CTP-16, CTP- | | Mutiple cancers |
| | 21 | | |
| Anti collagen | HU177; | | Multiple cancers |
| Types 1-V | HUIV26; | | |
| | XL313 | | |
| Anti-CD46 | | Crucell/J&J | Mutiple cancers |
| Anti-17A-1 | Edrecolomab | Panorex | Colorectal cancer |
| Anti-HM1.24 | AHM | | Multiple myeloma |
| Anti-CD38 | Anti-CD38 | | Multiple myeloma |
| Anti-IL15 | HuMax | | Lymphoma |
| Receptor | lymphoma | | |
| Anti-IL6 | B-E8 | | Lymphoma |
| Anti-TRAIL-R1 | TRM-1 | | Mutiple cancers |
| Anti-VEGF2 | | | Mutiple cancers |
| Anti-BlyS | Lymphostat | | Mutiple cancers |
| Anti-SCLC, CEA | Pentacea | | Lung cancer |
| and DTPA | | | |
| Anti-CD52 | CAMPATH | | Leukemia, Lymphoma |
| Anti-Lewis Y | IGN311 | | Epithelial cancers |
| antigen | | | |
| Anti-VE cadherin | E4G10 | | Mutiple cancers |
| Anti-CD56 | BB10901, | | Colorectal, lung cancer |
| | huN901DC1 | | |
| Anti- | Cantuzumab | | Colorectal, lung, pancreatic cancer |
| mertansine/mucine | | | |
| Anti-AFP | AFP-cide | | Liver cancer |
| Anti-CSAp | Mu-9 | | Colorectal cancer |
| Anti-CD30 | MDX-060 | | Melanoma, Hodgkins Disease |
| Anti-PSMA | MDX-070 | | Prostate cancer |
| Anti-CD15 | MDX-11 | | Leukemia |
| Anti-TAG72 | MDX-020 | | Colorectal cancer |
| Anti-CD19, CD3 | MT103 | | Lymphoma |
| bispecific | | | |
| Anti-mesothelin | SS1-PE38 | | Brain and overian cancer, |
| antigen | | | mesothelioma |
| Anti-DNA and | Cotara | | Colorectal, pancreatic, sarcoma, |
| histones | | | brain and other cancers |
| Anti-a5B1 integrin | Anti-a5 B1 | | Multiple cancers |
| Anti-p97 | SGN17/19 | | Melanoma |
| Anti-CD5 | Genimune | | Leukemia, lymphoma |

Alloreactive Natural Killer Cells

As used herein, “donor” means the subject that is the natural source from which the natural killer cells are originally removed. Also as used herein, a “recipient” is the subject into which the natural killer cells will be introduced.

Major histocompatability complex antigens (also called human leukocyte antigens, HLA) are protein molecules expressed on the surface of cells that confer a unique antigenic identity to these cells. MHC/HLA antigens are target molecules that are recognized by certain immune effector cells (T-cells and natural killer (NK) cells) as being derived from the same source of hematopoietic reconstituting stem cells as the immune effector cells (“self”) or as being derived from another source of hematopoietic reconstituting cells (“non-self”).

Natural killer (NK) cells are a sub-population of lymphocytes, involved in non-conventional immunity. NK cells can be obtained by various techniques known in the art, such as from blood samples, cytapheresis, collections, etc. NK cells are negatively regulated by major histocompatibility complex (MHC) class I-specific inhibitory receptors (Kärre et al., 1986; Öhlén et al, 1989; the disclosure of which is incorporated herein by reference). In humans, receptors termed killer Ig-like receptors (KIRs) recognize groups of HLA class I alleles. Although KIRs and other class-I inhibitory receptors (Moretta et al, 1997 ; Valiante et al, 1997a; Lanier, 1998; the disclosure of which is incorporated herein by reference) may be co-expressed by NK cells, in any given individual's NK repertoire there are cells that express a single KIR and are blocked only by a specific class I allele group. Missing expression of the KIR ligand on mismatched allogeneic cells can therefore trigger NK cell alloreactivity (Ciccone et al, 1992a, 1992b ; Colonna et al, 1993a, 1993b; Bellone et al, 1993; Valiante et al, 1997b; the disclosure of which is incorporated herein by reference). During hematopoietic transplants, when the recipient's class I alleles do not block all donor NK cells, donor alloreactive NK clones can be generated (Ruggeri et al, 1999; the disclosure of which is incorporated herein by reference).

Characteristics and biological properties of NK cells include the expression of surface antigens including CD16, CD56 and/or CD57, and the absence of the alpha/beta or gamma/delta TCR complex expressed on the cell surface; the ability to bind to and kill cells that fail to express “self” MHC/HLA antigens by the activation of specific cytolytic enzymes; the ability to kill tumor cells providing that tumor cells express a NKR-ligand; the ability to release protein molecules called cytokines that stimulate or inhibit the immune response; and the ability to undergo multiple rounds of cell division and produce daughter cells with similar biologic properties as the parent cell. Properties of monocytes include the ability to engulf bacteria and “non-self” cells (phagocytosis); the elaboration of cytokines that stimulate T cells and NK cells; the release of molecules that cause inflammation; and the presentation of antigens to T cells. By “active” NK cell is intended fully biologically active NK cells, more particularly NK cells having the capacity of lysing target cells. More particularly, “active” NK cells refer to an ex vivo cultures or expanded NK cell population, more preferably a NK cell population treated or cultured in vitro or ex vivo in the presence of a cytokine such as an interleukin, more preferably IL-2. For instance, an “active” NK cell is able to kill cells that express a NKR-ligand and fail to express “self” MHC/HLA antigens (KIR-incompatible cells).

Inhibition of natural killer (NK) cell lysis is signaled through specific receptors which bind to polymorphic determinants of major histocompatibility complex (MHC) class I molecules or HLA. Some receptors are a family of Ig-like molecules known as killer cell inhibitory receptors (KIR).

As used herein, alloreactive NK cells refer to NK cells which do not express a KIR (killer cell inhibitory receptor) able to bind a MHC/HLA antigen of the recipient (a KIR incompatibility in the donor-vs-recipient direction). More particular, said alloreactive NK cells are not able to bind one of the HLA-A, HLA-B or HLA-C antigen in the host, preferably the HLA-B or HLA-C antigen. The KIRs with two Ig domains (KIR2DL) identify HLA-C allotypes: KIR2DL2 (formerly designated p58.1) or the closely related gene product KIR2DL3 recognizes an epitope shared by group 1 HLA-C allotypes(Cw1, 3, 7, and 8), whereas KIR2DL1 (p58.2) recognizes an epitope shared by the reciprocal group 2 HLA-C allotypes(Cw2, 4, 5, and 6). One KIR with three Ig domains KIR3DL1 (p70) recognizes an epitope shared by HLA-Bw4 alleles. Finally, a homodimer of molecules with three Ig domains KIR3DL2 (p140) recognizes HLA-A3 and -A11.

The most interesting KIRs identify HLA-C allotypes. Indeed, only two KIRs, namely KIR2DL2 or KIR2DL3, and KIR2DL1, are sufficient for covering most of the HLA-C allotypes, respectively group 1 HLA-C allotypes and group 2 HLA-C allotypes.

KIR genes, each expressed by some of the individual's NK cells, vary considerably among individuals. It is believed that during development each NK cell precursor makes a random choice of which KIR genes it will express, and the different combinations of HLA class I molecules select NK cells that express receptors for self HLA class I. Consequently, the NK cells from any given individual will be alloreactive toward cells from others which lack their KIR ligands and, conversely, will be tolerant of cells from another individual who has the same or additional KIR ligands.

Therefore, the alloreactive NK cells are derived from a donor, more particularly an alloreactive donor, selected for having mismatch with the recipient for at least one antigen of the three major HLAs, preferably those of the HLA-C and HLA-B. For example, if the recipient presents a group 1 HLA-C allotype, the donor has a group 2 HLA-C allotype. Reciprocally, for a recipient having a group 2 HLA-C allotype, the donor is selected such that it presents a group 1 HLA-C allotype. In an additional example, for a recipient having a group Bw4 HLA-B allotype, the donor is selected such that it does not present a group Bw4 HLA-B allotype. Reciprocally, for a recipient who has not a group Bw4 HLA-B allotype, the donor is selected such that it presents a group Bw4 HLA-B allotype.

Remarkably, when donor-recipient haplo-mismatched pairs were also KIR ligand mismatched (as in this example), 100% of the donors tested had (at least some) alloreactive NK cells in their repertoires. Therefore, in a population of NK cells from an alloreactive donor, 5-50% of NK cells are alloreactive.

The alloreactive NK cells are prepared from a donor by different techniques which are known by the skilled person. More particularly, these cells can be obtained by different isolation and enrichment methods using peripheral blood mononuclear cells (lymphoprep, leucapheresis, etc. . . . ). These cells can be prepared by Percoll density gradients (Timonen et al., 1982; the disclosure of which is incorporated herein by reference), by negative depletion methods (Zarling et al., 1981; the disclosure of which is incorporated herein by reference) or by FACS sorting methods (Lanier et al., 1983; the disclosure of which is incorporated herein by reference). These cells can also be isolated by column immunoadsorption using an avidine-biotin system (Handgretinger et al., 1994; the disclosure of which is incorporated herein by reference) or by immunoselection using microbeads grafted with antibodies (Geiselhart et al., 1996-97; the disclosure of which is incorporated herein by reference). It is also possible to use combinations of these different techniques, optionally combined with plastic adherence methods. For example, the alloreactive NK cells can be prepared by providing blood mononuclear cells depleted of T cells from the donor, activating said cells with phytohemagglutinin (PHA) and culturing said cells with interleukin (IL)-2 and irradiated feeder cells. Optionally, the population of NK cells can be tested for the alloreactivity against the recipient cells. Optionally, said NK cells can be cloned and each clone is tested for the alloreactivity against the recipient cells. Optionally, the NK cell clones presenting the alloreactivity are pooled. The alloreactivity is tested by standard 51 Cr release cytotoxicity against recipient PHA lymphoblasts, or Epstein-Barr virus transformed B lymphoblastoid cell lines.

Therefore, the alloreactive NK cells according to the present invention can be prepared by a method comprising: a) providing NK cells from an alloreactive donor; b) activating said NK cells with IL-2; c) collecting the active NK cells resulting from step b). Optionally, said method comprises an additional step of testing the alloreactivity of the NK cells collected from step c) against the recipient cells. Alternatively, the alloreactive NK cells according to the present invention can be prepared by a method comprising: a) providing NK cells from an alloreactive donor; b) isolating or cloning said NK cells; c) activating said NK cells with IL-2; d) testing the alloreactivity of the NK cells resulting from step c) against the recipient cells; and, optionally, e) pooling the alloreactive NK cells. NK cells can be further expanded in vivo or in vitro.

In a first embodiment, said alloreactive NK cells contain essentially only alloreactive NK cells. In an alternative embodiment, said alloreactive NK cells refers to a population of NK cells prepared from an alloreactive donor. In this case, said population comprises both alloreactive and non-alloreactive NK cells. Preferably, this NK cells population comprises at least 5% of alloreactive NK cells, more preferably at least 20% of alloreactive NK cells, still more preferably at least 30% of alloreactive NK cells.

Composition and Administration

The invention concerns a composition comprising alloreactive donor-vs-recipient natural killer cells and a therapeutic antibody, the use of said composition for increasing the efficiency of the therapeutic antibody, for increasing ADCC in a subject treated with a therapeutic antibody, or for treating a subject having disease, more particularly disease requiring the depletion of the targeted cells, preferably the diseased cells such as virally-infected cells, tumor cells or other pathogenic cells, including allogenic immunocompetent cells. Preferably, the disease is selected from the group consisting of a cancer, an auto-immune disease, an inflammatory disease, a viral disease. The disease also concerns a graft rejection, more particularly allograft rejection, and graft versus host disease (GVHD).

Said therapeutic antibody can be bound by CD16, preferably through its Fc region. Preferably, said therapeutic antibody has a human or non human primate IgG1 or an IgG3 Fc portion, particularly a monoclonal antibody or a fragment thereof, further preferably a human, humanized or chimeric antibody or a fragment thereof, for insance rituximab.

Preferably, said composition is enriched in alloreactive natural killer cells regarding to the non-alloreactive natural killer cells content. Said composition comprises at least 5% of alloreactive NK cells among its NK cells content, preferably at least 20 or 30%, more preferably at least 40 or 50%, still more preferably at least 60, 70 or 90%. Optionally essentially all the NK cells comprised in said composition are allorective against the recipient cells.

Compositions of this invention may comprise any pharmaceutically acceptable carrier or excipient, typically buffer, isotonic solutions, aqueous suspension, optionally supplemented with stabilizing agents, preservatives, etc. Typical formulations include a saline solution and, optionally, a protecting or stabilizing molecule, such as a high molecular weight protein (e.g., human serum albumin).

According to the methods and compositions of the present invention, active alloreactive NK cells and therapeutic antibodies are administered in an efficient amount.

The efficient amount of alloreactive NK cells administered to the recipient can be between about 0.05 10⁶ and about 100 10⁶ cells/kg of recipient's body weight. Subranges of pure alloreactive NK cells are also provided, for example, about 0.05 10⁶ to 5 10⁶ cells/kg of recipient's body weight, about 5 10⁶ to 10 10⁶ cells/kg of recipient's body weight, about 10 10⁶ to 50 10⁶ cells/kg of recipient's body weight, about 50 10⁶ to 100 10⁶ cells/kg of recipient's body weight. Preferably, the amount of alloreactive NK cells administered to the recipient is comprised between 5 10⁶ to 15 10⁶ cells/kg of recipient's body weight.

The efficient amount of therapeutic antibodies administered to the recipient can be between about 0.1 mg/kg and about 50 mg/kg, preferably. The efficient amount of antibody depends however of the form of the antibody (whole Ig, or fragments), affinity of the mAb and pharmacokinetics parameter that must be determined for each particular antibodies.

In an important embodiment of the invention, the use of the present alloreactive NK cells can allow therapeutic efficacy to be achieved with reduced doses of therapeutic antibodies. The use (e.g., dosage, administration regimen) of therapeutic antibodies can be limited by side effects, e.g., in the case of rituximab, fever, headaches, wheezing, drop in blood pressure, and others. Accordingly, while in many patients a standard dose of the therapeutic antibodies will be administered in conjunction with the herein-described alloreactive NK cells (i.e., the recommended dose in the absence of any other compounds), thereby enhancing the efficacy of the standard dose in patients needing ever greater therapeutic efficacy, in other patients, e.g., those severely affected by side effects, the administration of the present compounds will allow therapeutic efficacy to be achieved at a reduced dose of therapeutic antibodies, thereby avoiding side effects. In practice, a skilled medical practitioner will be capable of determining the ideal dose and administrative regimen of the therapeutic antibody and the alloreactive NK cells for a given patient, e.g. the therapeutic strategy that will be most appropriate in view of the particular needs and overall condition of the patient. Numerous references are available to guide in the determination of proper dosages, for both the therapeutic antibodies and the alloreactive NK cells, e.g., Remington: The Science and Practice of Pharmacy, by Gennaro (2003), ISBN: 0781750253; Goodman and Gilmans The Pharmacological Basis of Therapeutics, by Hardman, Limbird & Gilman (2001), ISBN: 0071354697; Rawlins E. A., editor, “Bentley's Textbook of Pharmaceutics”, London: Bailliere, Tindall and Cox, (1977); and others.

In one embodiment, a medical practitioner can gradually lower the amount of the therapeutic antibody given in conjunction with the administration of alloreactive NK cells; either in terms of dosage or frequency of administration, and monitor the efficacy of the therapeutic antibody; e.g. monitor NK cell activity; monitor the presence of target cells in the patient, monitor various clinical indications, or by any other means, and, in view of the results of the monitoring, adjust the relative concentrations or modes of administration of the therapeutic antibodies and/or alloreactive NK cells to optimize therapeutic efficacy and limitation of side effects.

The composition according to the present invention may be injected directly to a subject, typically by intra-venous, intra-peritoneal, intra-arterial, intramuscular or transdermic route. Several monoclonal antibodies have been shown to be efficient in clinical situations, such as Rituxan (Rituximab) or Xolair (Omalizumab), and similar administration regimens (i.e., formulations and/or doses and/or administration protocols) may be used with the composition of this invention.

Furthermore, the compositions of this invention may further comprise or may be used in combination with other active agents or therapeutic programs such as chemotherapy or other immunotherapies, either simultaneously or sequentially.

Advantageously, the method of the invention further comprises one or several injections of an effective amount of a molecule, preferably a cytokine, capable of stimulating NK cell activity. This molecule can be used in combination with the compounds that block an inhibitory receptor or stimulate an activating receptor of a NK cell and thereby result in an even greater augmentation of ADCC and efficacy of therapeutic antibodies. Said molecule or cytokine may for example increase NK cell proliferation, NK cell cytokine production, intracellular free calcium levels, or the ability of NK cells to lyse target cells in a redirected killing assay. The invention therefore also provides a method of treatment of a disease in a subject in need thereof comprising: a) administering to said subject a compound, preferably an antibody or a fragment thereof, that blocks an inhibitory receptor or stimulates an activating receptor of a NK cell; b) administering to said subject a therapeutic antibody, and (c) administering to said subject a cytokine capable of stimulating NK cell activity. Examples of such cytokines include interleukins such as IL2 (Research Diagnostics, NJ, RDI-202), IL12 (Research Diagnostics, NJ, DI-212), IL15 (Research Diagnostics, NJ, RDI-215), IL21 (Asano et al, FEBS Lett. 2002;528:70-6) or a combination thereof.

The cytokine can be administered according to any suitable administration regimen, and may be administered before, simultaneously and/or after administration of the compound which blocks an inhibitory receptor or stimulates an activating receptor of a NK cell, and before, simultaneously and/or after administration of therapeutic antibody. In a typical example, the cytokine is administered daily for a period of 5-10 days, the cytokine(s) being first injected on the same day as the first injection of the compound which blocks an inhibitory receptor or stimulates an activating receptor of a NK cell. Said method preferably comprises one or two injections/day of cytokine(s) by subcutaneous route.

The dosage of the cytokine will be chosen depending on the condition of the patient to be treated. In preferred examples, a relatively low dose of cytoline can be used. For example, an effective dose of a is typically lower than 1 million units/square meters/day of cytokine(s), when the cytokine-containing pharmaceutical composition is used for daily subcutaneous injection. In a preferred example, IL-2 is injected subcutaneously at daily doses below 1 million units/m² for 5 to 10 days. Further detail of the use of cytokines is described in International Patent publication no. PCT/EP/0314716 and U.S. patent application no. 60/435,344 titled “Pharmaceutical compositions having an effect on the proliferation of NK cells and a method using the same”, the disclosures of which are incorporated herein by reference.

According to the invention the host patient is conditioned prior to the transplantation of the allogeneic graft. Conditioning may be carried out under sublethal, lethal or supralethal conditions, for example by total body irradiation (TBI) and/or by treatment with myelo-reductive or myelo-ablative and immunosuppressive agents. According to standard protocols, a lethal dose of irradiation is within the range of 7-9,5 Gy TBI, a sublethal dose is within the range of 3-7 Gy TBI and a supralethal dose is within the range of 9,5-16 Gy TBI.

Any immunosuppressive agent used in transplantation to control the rejection, or a combination of such agents, can be used according to the invention, such as prednisone, methyl prednisolone, azathioprine, cyclophosphamide, cyclosporine, monoclonal antibodies against T-cells, e.g. OKT3, and antisera to human lymphocytes (antilymphocyte globulin—ALS) or to thymus cells (antithymocyte globulin—ATG). Examples of myelo-ablative agents that can be used according to the invention are busulphan, dimethyl myleran and thiotepa.

### EXAMPLES

Preparation of human NK clones. Blood mononuclear cells depleted of T cells by negative anti-CD3 immuno-magnetic selection (Miltenyi) are plated under limiting-dilution conditions, activated with phytohemagglutinin (PHA) (Biochrom KG, Berlin, Germany), and cultured with interleukin (IL)-2 (Chiron B.V., Amsterdam, Netherlands) and irradiated feeder cells. Cloning efficiencies are equivalent in all donors and range between 1 in 5 and 1 in 10 plated NK cells. Cloned NK cells are screened for alloreactivity by standard ⁵¹Cr release cytotoxicity against Epstein-Barr virus-transformed B lymphoblastoid cell lines of known HLA type at an effector to target ratio of 10:1. Clones exhibiting=30% lysis were scored as alloreactive. As a rule, clones either exhibit <5% or >40% lysis.

Cytotoxicity Experiments.

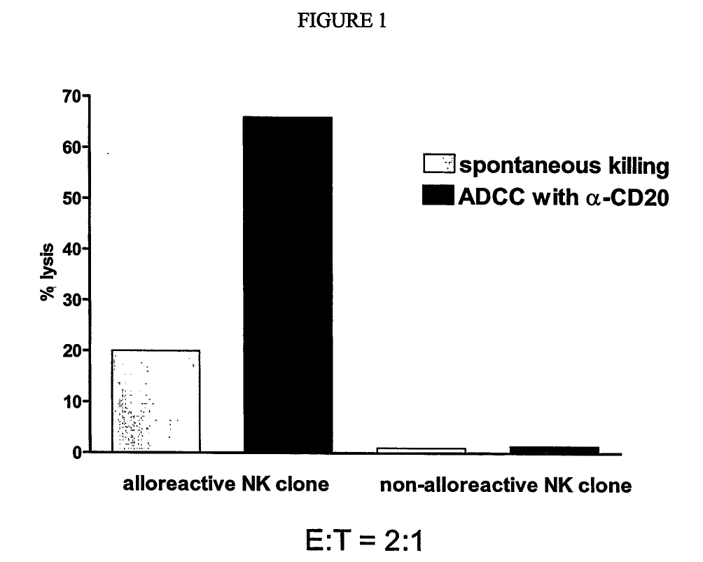

The cytolytic activity of NK clones was assessed by a standard 4 hr ⁵¹Cr release assay, in which effector NK cells were tested on Cw3 or Cw4 positive EBV cell lines (CD20 positive), known for their sensitivity to NK cell lysis. All the targets are used at 5000 cells per well in microtitration plate and the Effector (NK cell clone) on target ratio is indicated in the FIG. 1. In certain experiments, the therapeutic chimeric anti CD20 rituximab (Rituxan, Idec) is added at 5 μg/ml is added to the effector target mixture.

This experiment showed that only the alloreactive NK cells present the capacity to lyse the cells. The antibody alone had no effect on the cell lysis when used with non alloreactive NK. The incubation of the cells with both alloreactive NK cells and the antibody increase by about three folds the rate of lysis, then the cytotoxicity of the alloreactive NK cells.

Preparation of human EBV line-engrafted mice. 3.5 Gy-irradiated NOD-SCID mice, (Charles River Italia, Calco, Italy) are given 2×10⁷ human EBV line cells. These mice die within 2 weeks and display extensive bone marrow and spleen infiltration with EBV line cells.

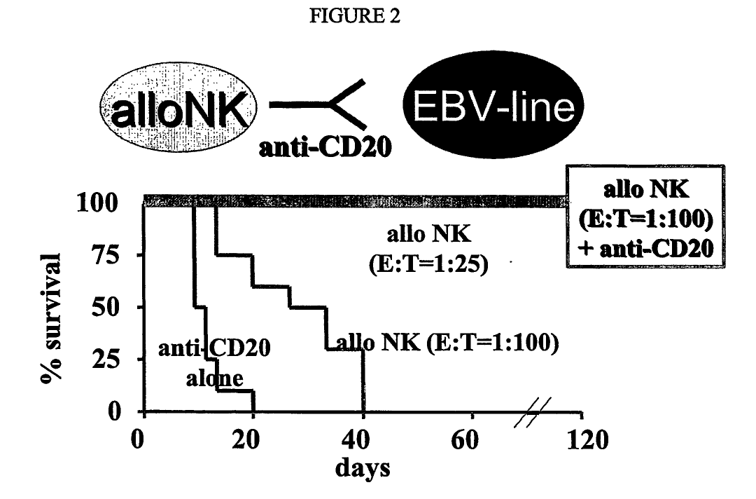

Rescue of mice by alloreactive NK cells+ADCC. Six hours of the infusion of the EBV line mice are given human NK cells that are alloreactive against the EBV line (or control non-alloreactive cells) at various E:T ratios, with or without the clinically available humanized anti-CD20 antibody at the same per Kg dosage as in humans. Alloreactive NK cells alone rescue 100% of mice when they are used at an E:T ratio of 1:25, but not at lower ratios such as 1:100. When we combine the 1:100 ratio of alloreactive NK cells with the anti-CD20 antibody, we observe a four-fold increase in the therapeutic potential of alloreactive NK cells (100% of mice that received the 1:100 ratio of alloreactive NK cells survived). (FIG. 2)

### REFERENCES

Bellone G, Valiante N M, Viale O, Ciccone E, Moretta L, Trinchieri G (1993) <<Regulation of hematopoiesis in vitro by alloreactive natural killer cell clones”: J Exp Med 177:1117-25

Ciccone E, Pende D, Viale O, Di Donato C, Tripodi G, Orengo A M, Guardiola J, Moretta A, Moretta L (1992a) “Evidence of a natural killer (NK) cell repertoire for (allo) antigen recognition: definition of five distinct NK-determined allospecificities in humans”: J Exp Med 175:709-18

Ciccone E, Pende D, Viale O, Than A, Di Donato C, Orengo A M, Biassoni R, Verdiani S, Amoroso A, Moretta A, et al. (1992b) “Involvement of HLA class I alleles in natural killer (NK) cell-specific functions: expression of HLA-Cw3 confers selective protection from lysis by alloreactive NK clones displaying a defined specificity (specificity 2).” J Exp Med 176:963-71

Colonna M, Brooks E G, Falco M, Ferrara G B, Strominger J L (1993a) “Generation of allospecific natural killer cells by stimulation across a polymorphism of HLA-C” Science 260:1121-4

Colonna M, Borsellino G, Falco M, Ferrara G B, Strominger J L (1993b) “HLA-C is the inhibitory ligand that determines dominant resistance to lysis by NK1- and NK2-specific natural killer cells” Proc Natl Acad Sci USA 90:12000-4

Geiselhart A, Neu S, Buchholz F, Lang P, Niethammer D, Handgretinger R (1996-97) “Positive selection of CD56+lymphocytes by magnetic cell sorting” Nat Immun 15:227-33

Handgretinger R, Welte B, Dopfer R, Niethammer D (1994) “Rapid method for purification of CD56+ natural killer cells with preferential enrichment of the CD56bright+ subset” J Clin Lab Anal 8:443-6

Karre K, Ljunggren H G, Piontek G, Kiessling R, (1986) “Selective rejection of H-2-deficient lymphoma variants suggests alternative immune defence strategy” Nature 319:675-8

Karre K (2002) “Immunology. A perfect mismatch” Science 295:2029-31

Lanier L L, Le A M, Phillips J H, Warner N L, Babcock G F (1983) “Subpopulations of human natural killer cells defined by expression of the Leu-7 (HNK-1) and Leu-1 1 (NK-15) antigens” J Immunol 131:1789-96

Lanier L L (1998) <<NK cell receptors” Annu Rev Immunol 16:359-93

Moretta A, Moretta L (1997) <<HLA class I specific inhibitory receptors” Curr Opin Immunol 9:694-701

Ohlen C, Kling G, Hoglund P, Hansson M, Scangos G, Bieberich C, Jay G, Karre K (1989) “Prevention of allogeneic bone marrow graft rejection by H-2 transgene in donor mice” Science 246:666-8

Ravetch, J V and Bolland, S (2001) IgG Fc receptors. Annu Rev Immunol 19,275-290.

Ruggeri L, Capanni M, Casucci M, Volpi I, Tosti A, Perruccio K, Urbani E, Negrin R S, Martelli M F, Velardi A (1999) “Role of natural killer cell alloreactivity in HLA-mismatched hematopoietic stem cell transplantation” Blood 94:333-9

Ruggeri, L., Capanni, M., Urbani, E., Perruccio, K., Shlomchik, W. D., Tosti, A., Posati, S., Rogaia, D., Frassoni, F., Aversa, F., et al. (2002). Effectiveness of donor natural killer cell alloreactivity in mismatched hematopoietic transplants. Science 295, 2097-2100

Timonen T, Reynolds C W, Ortaldo J R, Herberman R B, (1982) “Isolation of human and rat natural killer cells” J. Immunol. Methods, 51, 269-77

Valiante N M, Lienert K, Shilling H G, Smits B J, Parham P (1997a) <<Killer cell receptors: keeping pace with MHC class I evolution” Immunol Rev 155:155-64

Valiante N M, Parham P (1997b) <<Natural killer cells, HLA class I molecules, and marrow transplantation” Biol Blood Marrow Transplant 3:229-35

Zarling J M, Clouse K A, Biddison W E, Kung P C, (1981) “Phenotypes of human natural killer cell populations detected with monoclonal antibodies” J. Imunol. 127, 2575-80

1-33. (canceled)

34. A method of treatment of a disease in a human subject in need thereof, comprising:
a) administering to said subject alloreactive natural killer cells; and
b) administering to said subject a therapeutic antibody which can be bound by CD16.

35. The method according to claim 34, wherein said therapeutic antibody has a human or non human primate IgG1 or an IgG3 Fc portion.

36. The method according to claim 35, wherein said therapeutic antibody is a monoclonal antibody or a fragment thereof.

37. The method according to claim 35, wherein said therapeutic antibody is a chimeric, humanized, or human antibody or a fragment thereof.

38. The method according to claim 37, wherein said therapeutic antibody is rituximab.

39. The method according to claim 34, wherein said alloreactive natural killer cells comprise at least 5% of alloreactive donor-vs-recipient natural killer cells.

40. The method according to claim 39, wherein said alloreactive natural killer cells comprise at least 30% of alloreactive donor-vs-recipient natural killer cells.

41. The method according to claim 40, wherein said alloreactive natural killer cells comprise at least 50% of alloreactive donor-vs-recipient natural killer cells.

42. The method according to claim 41, wherein said alloreactive natural killer cells comprise at least 90% of alloreactive donor-vs-recipient natural killer cells.

43. The method according to claim 34, wherein said therapeutic antibody and said alloreactive natural killer cells are administered into said subject simultaneously.

44. The method according to claim 34, wherein said therapeutic antibody are administered into said subject prior to said alloreactive natural killer cells.

45. The method according to claim 34, wherein said disease is a cancer, infectious or immune disease.

46. A pharmaceutical composition comprising a therapeutic antibody, which can be bound by CD 16, and alloreactive natural killer cells.

47. The composition according to claim 46, wherein said therapeutic antibody has a human or non human primate IgG1 or an IgG3 Fc portion.

48. The composition according to claim 47, wherein said therapeutic antibody is a monoclonal antibody or a fragment thereof.

49. The composition according to claim 47, wherein said therapeutic antibody is a human, humanized or chimeric antibody or a fragment thereof.

50. The composition according to claim 49, wherein said therapeutic antibody is rituximab.

51. The composition according to claim 46, wherein said alloreactive natural killer cells comprise at least 5% of alloreactive donor-vs-recipient natural killer cells.

52. The composition according to claim 51, wherein said active alloreactive natural killer cells comprise at least 30% of alloreactive donor-vs-recipient natural killer cells.

53. The composition according to claim 52, wherein said active alloreactive natural killer cells comprise at least 50% of alloreactive donor-vs-recipient natural killer cells.

54. The composition according to claim 53, wherein said active alloreactive natural killer cells comprise at least 90% of alloreactive donor-vs-recipient natural killer cells.

55. A method of increasing antibody mediated cellular cytotoxicity (ADCC) in a subject receiving therapeutic antibody treatment or increasing the efficiency of a therapeutic antibody treatment in a subject, wherein said antibody can be bound by CD16 and said method comprises administering to said subject prior to, simultaneously or after the administration of said therapeutic antibody an amount of alloreactive natural killer cells sufficient to increase ADCC.

56. The method according to claim 55, wherein said therapeutic antibody has a human or non human primate IgG1 or an IgG3 Fc portion.

57. The method according to claim 56, wherein said therapeutic antibody is a monoclonal antibody or a fragment thereof.

58. The method according to claim 57, wherein said therapeutic antibody is a human, humanized or chimeric antibody or a fragment thereof.

59. The method according to claim 58, wherein said therapeutic antibody is rituximab.

60. The method according to claim 55, wherein said alloreactive natural killer cells comprise at least 5% of alloreactive donor-vs-recipient natural killer cells.

61. The method according to claim 60, wherein said alloreactive natural killer cells comprise at least 30% of alloreactive donor-vs-recipient natural killer cells.

62. The method according to claim 61, wherein said alloreactive natural killer cells comprise at least 50% of alloreactive donor-vs-recipient natural killer cells.

63. The method according to claim 62, wherein said alloreactive natural killer cells comprise at least 90% of alloreactive donor-vs-recipient natural killer cells.

64. The method according to claim 55, wherein said therapeutic antibody and said alloreactive natural killer cells are administered into said subject simultaneously.

65. The method according to claim 55, said therapeutic antibody are administered into said subject prior to said alloreactive natural killer cells.
